(19) United States
(12) Patent Application Publication
(10) Pub. No.: US 2008/0160536 A1
(43) Pub. Date: Jul. 3, 2008

(54) Methods For Detecting Calcium Ion Influx

(75) Inventors: Peter Lockyer, Cambridge (GB); Julie Lockyer, Dorset (GB)

Correspondence Address: GOODWIN PROCTER LLP;PATENT ADMINISTRATOR, EXCHANGE PLACE, BOSTON, MA, 02109-2881, US

(73) Assignee: BABRAHAM INSTITUTE, Cambridge, Cambridgeshire (GB)

(21) Appl. No.: 10/593,852

(22) PCT Filed: Mar. 23, 2005
(86) PCT No.: PCT/GB2005/050042

§ 371 Date: Sep. 10, 2007

(30) Foreign Application Priority Data
Mar. 23, 2004 (GB) 0406479.6

(51) Int. Cl. G01N 33/53

(52) U.S. Cl. 435/7.1; 435/29

(57) ABSTRACT

A method for detecting influx of calcium ions into a eukaryotic cell comprising providing a eukaryotic cell having a detectable reporter capable of translocation from the cytosol to associate with the plasma membrane in response to an influx of calcium ions, and, monitoring association of the detectable report with the plasma membrane and/or a decrease in the detectable report in the cytosol. The detectable report is preferably CAPRI or a derivative thereof and is preferably labelled with a fluorescent marker.

### TECHNICAL FIELD

The invention relates to live, whole cell assays for detecting calcium ion (Ca²⁺) influx using a detectable reporter, particularly a fluorescent reporter. The methods are useful for detection of compounds that modulate calcium influx and can be performed in high throughput screening format.

### BACKGROUND TO THE INVENTION

Calcium influx into the cell from the extracellular medium is vital for processes such as muscle contraction, secretion and gene activation. Calcium influx is mediated via calcium influx channels which can be divided into two groups on the basis of their activation mechanism: voltage-gated calcium channels and non-voltage-gated calcium-permeable calcium channels. Calcium influx can be stimulated or inhibited by factors that act on the calcium ion channel directly, or indirectly where modulation of a receptor results in a signal that acts on a calcium ion channel.

Ion channels play an important role in numerous cell types and occur as large families of related genes. Ion channels, such as store-operated calcium channels, receptor-operated and voltage-gated calcium channels are important selective tissue-specific targets for drug discovery. Currently more than a dozen ion channel drugs are marketed for the treatment of cardiovascular disease, diabetes, epilepsy and pain. Thus, there is interest in developing cell-based assays for screening compounds that modulate the activity of ion channels and receptors associated with ion channels.

With the demand for screening large compound collections against an increasing number of therapeutic targets, there is a demand for improved assays, especially cell-based assays and for such assays in automated, high throughput formats.

Technologies currently employed for ion channel screening include binding assays, fluorometric imaging and voltage sensing.

Whole cell functional assays are known for identification of compounds that act at plasma membrane ion channels, or receptors such as G-protein coupled receptors (GPCR) or receptor tyrosine kinases (RTKs), and trigger calcium influx. The rapid increase in intracellular calcium concentration triggered by stimulation of these ion channels and receptors is detected using intracellular calcium probes; known probes include fluorescent dyes, or calcium-binding proteins. To detect the rapid, transient changes in intracellular calcium concentration on stimulation, a calcium influx stimulus is provided to cells and a change in fluorescence or luminescence from the calcium probe is detected. A problem with many current methods is that the signal generated is of short duration and this restricts use of these assays in high throughput systems.

There have been attempts to delay or prolong the calcium signalling response to provide a greater temporal window for detection of calcium influx. One approach to prolong the calcium signalling response is described in U.S. Pat. No. 6,514,709 in which an intracellular calcium chelating agent 1,2-bis(o-aminophenoxy)ethane-N,N,N′,N′-tetraacetic acid tetra(acetoxymethyl)ester (BAPTA-AM) was used to alter the kinetics of calcium signalling and delay or prolong the calcium signalling response.

A further problem is that assay methods using fluorescent Ca²⁺ dyes in cell lines expressing a Ca²⁺ channel of interest are not generally suitable for targeting to a specific sub-cellular compartment of interest, e.g. under the plasma membrane. The dyes are generally freely diffusible and give only a global indication of a Ca²⁺ change. Ca²⁺ dyes must be loaded into the cells, which can be done fairly consistently, but is inherently prone to differences between experiments. Generally, dyes do not report restricted spatial Ca²⁺ signals, e.g. extracellular Ca²⁺ influx induced by channel opening.

Another drawback of present assay methods is that intracellular agents used to detect calcium may adversely affect the behaviour of the cell, for example by buffering cytosolic calcium signals, and so the assay methods may not provide a true reflection of cellular events following calcium influx.

There is a desire to develop alternative whole cell assays using reporters sensitive to calcium influx.

The proteins RASAL and CAPRI (calcium promoted Ras inactivator) have been reported to be sensitive to increases in intracellular calcium due to ATP stimulation of calcium release from the intracellular calcium store. In resting cells RASAL and CAPRI are cytosolic and inactive. Following ATP stimulation, RASAL and CAPRI are translocated to the membrane; CAPRI is believed to undergo a conformation change and activation [1].

CAPRI is a member of the human GAP1 family of Ras GAPs (GAP1^{IP4BP}, GAP1^{m}, RASAL). The human GAP1 Ras GAPs have a similar domain structure comprising of tandem C2 domains (C2A and C2B), a central GAP-related domain (GRD) contiguous with a pleckstrin homology domain (PH domain) and Tec kinase homology domain (TH) near the C-terminus (FIG. 1). Within the human GAP1 family, RASAL [3] is most closely related to CAPRI with 59% identity at the primary amino acid sequence level.

Ras operates as a binary molecular switch, cycling between an inactive GDP-bound form and an active GTP-bound form at the plasma membrane, or other intracellular membranes such as the cytosolic face of the Golgi. The intrinsic GDP/GTP exchange and GTPase activity of Ras is slow, therefore control of GTPase cycling is modulated by guanine nucleotide exchange factors (GEFs) that promote the active GTP-bound state, and GTPase-activating proteins (GAPs) that promote the inactive GDP-bound state.

Activated CAPRI inhibits the Ras/mitogen-activated protein kinase (MAPK) pathway by enhancing the intrinsic GTPase activity of Ras, resulting in deactivation of Ras. Analysis of the spatio-temporal dynamics of CAPRI and RASAL indicates that calcium regulates Ras by a fast C2 domain-dependent translocation mechanism. Analysis of this mechanism has been carried out in a whole cell assay in which RASAL, CAPRI and CAPRI mutants tagged with a green fluorescent protein (GFP) were expressed. ATP-induced release of calcium ions from the intracellular store induced a rapid translocation of RASAL and CAPRI to the plasma membrane and activation of CAPRI. This recruitment of RASAL and CAPRI to the plasma membrane was detected because the GFP reporter resulted in acquisition of a fluorescent signal at the plasma membrane [1].

### STATEMENT OF INVENTION

The present invention provides a method for detecting influx of calcium ions into a eukaryotic cell comprising providing a eukaryotic cell having a detectable reporter capable of translocation from the cytosol to associate with the plasma membrane in response to an influx of calcium ions, and monitoring location of the detectable reporter within the cell.

The present invention provides a method for detecting influx of calcium ions into a eukaryotic cell comprising providing a eukaryotic cell having a detectable reporter capable of translocation from the cytosol to associate with the plasma membrane in response to an influx of calcium ions, and, monitoring association of the detectable reporter with the plasma membrane and/or a change in the amount of detectable reporter in the cytosol.

Both CAPRI and RASAL are Ca²⁺-triggered Ras (GAPs, they are located in the cytosol and are inactive in resting cells. Elevation of intracellular Ca²⁺ induces a rapid translocation of RASAL and CAPRI to the plasma membrane. This is mediated by tandem C2 domains (C2A C2B) that are known to be required for co-operative Ca²⁺/phospholipid-binding in other C2 domain Ca²⁺ sensor proteins.

All known C2 domain containing Ca²⁺ sensors that do not constitutively associate with cell membranes, e.g. through transmembrane domains, translocate to the membrane in phase with complex Ca²⁺ signals such as oscillations. Thus, RASAL and conventional protein kinase Cs (PKCs) oscillate on and off the membrane in concert with repetitive Ca²⁺ (and diacylglycerol in the case of PKC) signals. A major driver of complex oscillations in many cells is so-called Ca²⁺-induced Ca²⁺ release (CICR), a repetitive release of Ca²⁺ from intracellular Ca²⁺ stores.

When intracellular stores are depleted as Ca²⁺ is released into the cytosol there is a retrograde signal to the plasma membrane to open Ca²⁺ channels at the cell surface, so-called capacitative or store-operated Ca²⁺ entry (SOCE) calcium influx. This source of Ca²⁺ helps to replenish the intracellular Ca²⁺ store and maintain CICR. In addition, there are receptor-operated Ca²⁺ influx mechanisms and other non-store-operated Ca²⁺ entry (NSOCE) that operate in concert with, or in the absence of SOCE, in a stimulus-specific and cell type-specific manner. Unlike Ca²⁺ release, Ca²⁺ influx is not generally oscillatory.

It has now been found that RASAL and CAPRI are differentially tuned to Ca²⁺ signals. RASAL preferentially senses intracellular Ca²⁺ release, can oscillate between the cytosol and membrane in concert with complex Ca²⁺ signals, and therefore behaves as a frequency-modulated Ca²⁺ sensor. In contrast, CAPRI preferentially senses Ca²⁺ influx, is refractory to cytosolic Ca²⁺ oscillations, and therefore behaves like an analogue Ca²⁺ sensor of sustained Ca²⁺ influx. Unlike all other known ‘translocation sensors’ of Ca²⁺ signals, CAPRI does not sense oscillations of intracellular free Ca²⁺. Instead, CAPRI senses SOCE and is maintained at the plasma membrane for as long as SOCE occurs, even in the presence of repetitive changes in cytosolic Ca²⁺ levels such as baseline spiking and sinusoidal oscillations. Translocation of a CAPRI reporter from the cytosol to the plasma membrane and maintenance at that location indicates that Ca²⁺ influx, specifically, is occurring.

Thus CAPRI and CAPRI derivatives have been identified as novel preferential sensors of Ca²⁺ influx across the plasma membrane of cells.

Translocation of CAPRI to the plasma membrane is due to its preferential sensitivity to calcium influx rather than just release of calcium from the internal calcium store as initially thought. CAPRI and CAPRI derivatives can act as calcium influx specific reporters useful in assay methods for detection of calcium influx into eukaryotic cells.

In methods of the invention, it is preferred that the detectable reporter is or comprises CAPRI, or a derivative thereof which is capable of translocation to and association with the plasma membrane specifically in response to an influx of calcium ions, labelled with a detectable marker.

Methods of the invention can be applied to whole-cell functional assays, in particular to detect compounds that modulate calcium ion channels or that modulate receptors associated with calcium ion channels. The present invention includes methods for identifying compounds that specifically interact with receptor polypeptides such as G-protein coupled receptors (GPCRs) or receptor tyrosine kinases (RTKs). Compounds that interact with a receptor can stimulate or inhibit the activity of a receptor. The term “compounds” as used herein includes chemically synthesised molecules and includes biological molecules such as proteins and peptides, antigens, epitopes and mimics of epitopes.

The present invention provides method for identifying a compound capable of modulating influx of calcium ions into a eukaryotic cell comprising:
(a) providing a eukaryotic cell having a detectable reporter capable of translocation from the cytosol to associate with the plasma membrane in response to an influx of calcium ions,
(b) incubating the cell with a test compound and providing a stimulus for calcium ion influx, and,
(c) monitoring association of the detectable reporter with the plasma membrane and/or a decrease in the detectable reporter in the cytosol.

The stimulus for calcium ion influx may be provided before, during or after provision of the test compound to the cell.

Accordingly, the present invention provides method for identifying a compound capable of modulating influx of calcium ions into a eukaryotic cell comprising:
(a) providing a eukaryotic cell having a detectable reporter capable of translocation from the cytosol to associate with the plasma membrane in response to an influx of calcium ions,
(b) incubating the cell with a test compound, then
(c) providing a stimulus for calcium ion influx, and,
(d) monitoring association of the detectable reporter with the plasma membrane and/or a decrease in the detectable reporter in the cytosol.

In this aspect of the invention the cell is incubated with the test compound before calcium influx is stimulated.

Also provided is a method for identifying a compound capable of modulating influx of calcium ions into a eukaryotic cell comprising:
(a) providing a eukaryotic cell having a detectable reporter capable of translocation from the cytosol to associate with the plasma membrane in response to an influx of calcium ions,
(b) providing a stimulus for calcium ion influx,
(c) incubating the cell with a test compound,
(d) monitoring dissociation of the detectable reporter with the plasma membrane and/or an increase in detectable reporter in the cytosol.

In this aspect of the invention, calcium influx into the cell is stimulated and then the cell is exposed to the test compound. Ideally, the cell is exposed to the test compound during calcium ion influx and the rate of dissociation of the reporter from the membrane verses the rate observed in a control cell(s) not exposed to the test compound is assessed.

A compound capable of modulating influx of calcium ions is a compound that alters the behaviour of an ion channel or receptor and is also termed a modulator, a modulator may be an antagonist, inhibitor or blocker that, directly or indirectly, decreases calcium influx through an ion channel; alternatively the modulator can be an agonist or stimulator that, directly or indirectly, increases calcium influx through a calcium ion channel. As used herein, an “agonist” is a molecule that stimulates an activity of an ion channel or receptor; an “antagonist” is a molecule that inhibits or interferes with the activation of an ion channel or receptor. An “inhibitor” or “blocker” is a molecule prevents or reduces the opening of an ion channel or activation of a receptor.

The stimulus for calcium influx used in a method of the invention can be a stimulus or combination of stimuli for calcium influx, the stimulus can be one or more of the following:
(a) agonist-evoked intracellular calcium release from intracellular stores, which indirectly stimulates calcium influx through SOCE,
(b) agonist-evoked intracellular calcium release from intracellular stores, which stimulates calcium influx through NSOCE,
(c) agonist-evoked intracellular calcium release from intracellular stores, which stimulates calcium influx through SOCE and NSOCE,
(d) chemical treatment leading to store release that activates SOCE, e.g. using the Ca²⁺ pump inhibitor thapsigargin,
(e) an intracellular ligand that agonises a second messenger-operated plasma membrane Ca²⁺ channel,
(f) an agent or conditions that cause depolarisation thereby opening a voltage-gated channel,
(g) a stretch stimulus that opens a mechano-sensitive channel,
(h) a change in temperature sufficient to open a temperature sensitive Trp family channel,
(i) an agonist that opens a receptor-operated channel,
(j) an extracellular ligand that opens a ligand-gated channel,
(k) an intracellular or extracellular pH change that opens a redox-sensitive channel,
(l) a change in osmolarity that opens an osmolarity-sensitive channel.

Calcium ion channels can be opened by changes in lipid metabolism after phospholipase C activity induced by a GPCR or receptor tyrosine kinase (RTK) or by the products of phospholipase C activity such as diacyiglycerol (DAG).

A G protein-coupled receptor such as a purinergic receptor can be stimulated using ATP, this causes Ca²⁺ release from internal stores which eventually triggers a signal for calcium influx (SOCE).

Receptor tyrosine kinases (RTKs) are enzymes which catalyze the phosphorylation of tyrosine residues. RTKs are involved in cellular signaling pathways and regulate key cell functions such as proliferation, differentiation, anti-apoptotic signaling and neurite outgrowth. Unregulated activation of these enzymes, through mechanisms such as point mutations or over-expression, can lead to various forms of cancer as well as benign proliferative conditions. Oncogenes and proto-oncogenes involved in cancer are known to code for RTKs. The importance of RTKs in health and disease is further underscored by the existence of aberrations in RTK signaling occurring in inflammatory diseases and diabetes.

RTKs possess an extracellular ligand binding domain, a transmembrane domain and an intracellular catalytic domain. The transmembrane domain anchors the receptor in the plasma membrane, while the extracellular domains bind growth factors. Characteristically, the extracellular domains are comprised of one or more identifiable structural motifs, including cysteine-rich regions, fibronectin III-like domains, immunoglobulin-like domains, EGF-like domains, cadherin-like domains, kringle-like domains, Factor VII-like domains, glycine-rich regions, leucine-rich regions, acidic regions and discoidin-like domains.

The intracellular kinase domains of RTKs can be divided into two classes: those containing a stretch of amino acids separating the kinase domain and those in which the kinase domain is continuous. Activation of the kinase is achieved by ligand binding to the extracellular domain, which induces dimerization of the receptors. Receptors thus activated are able to autophosphorylate tyrosine residues outside the catalytic domain via cross-phosphorylation. The results of this auto-phosphorylation are stabilization of the active receptor conformation and the creation of phosphotyrosine docking sites for proteins which transduce signals within the cell. Signaling proteins which bind to the intracellular domain of receptor tyrosine kinases in a phosphotyrosine-dependent manner include RasGAP, PI3-kinase, phospholipase Cγ, phosphotyrosine phosphatase SHP and adaptor proteins such as Shc, Grb2 and Crk.

DAG has been shown to activate channels directly (TRPC3 and TRPC6) or through products of its breakdown, poly-unsaturated fatty acid metabolites such as arachidonic acid or linoleic acid (TRPL). TRPV channels can be activated using mechanical and physical stimuli such as low pH, heat and osmotic stress and ligands such as capsaicin and anadamide. Menthol and cold can be used to activate TRPM8 channels. In some cell types such as HeLa and HEK293, NSOCE can be activated directly by application of extracellular arachidonic acid through unknown channels (that may be TRP channels) termed arachidonate-regulated Ca²⁺ channels (ARC). Compounds other than thapsigargin that inhibit Ca²⁺ pumps leading to the depletion of intracellular store Ca²⁺ and reciprocal activation of SOCE include cyclopiazonic acid (CPA) and 2,5-di(tert-butyl)hydroquinone (BHQ).

The invention includes a method for identifying a compound that modulates calcium ion influx comprising:
(a) providing a eukaryotic cell having a detectable reporter capable of translocation from the cytosol to associate with the plasma membrane in response to an influx of calcium ions,
(b) monitoring association of the reporter with the membrane, and/or loss of reporter from cytosol while
   (i) incubating the cell in conditions that stimulate calcium influx, then
   (ii) introducing a test compound to the incubation media, and,
(c) comparing the association of the reporter with the membrane in the presence of the test compound with the association of the reporter with the membrane in the absence of the test compound,
   wherein a change in the association of the reporter with the membrane in the presence of the test compound indicates that the test compound modulates calcium influx.

In this aspect of the invention, calcium influx into the cell is stimulated and then the cell is exposed to the test compound. In a preferred embodiment, the cell is exposed to the test compound during calcium ion influx and the rate of dissociation of the reporter from the membrane verses the rate observed in a control cell(s) not exposed to the text compound is assessed.

The present invention provides methods useful as assays for identification of compounds that are receptor inhibitors (antagonists) or receptor activators (agonists).

In particular, the invention provides a method for identifying a compound that inhibits calcium ion influx comprising:
(a) providing a eukaryotic cell having a detectable reporter capable of translocation from the cytosol to associate with the plasma membrane in response to an influx of calcium ions,
(b) monitoring association of the reporter with the membrane while
   (i) incubating the cell in conditions that stimulate calcium influx, then,
   (ii) introducing a test compound to the incubation media, and,
(c) comparing the association of the reporter with the membrane in the presence and absence of the test compound,
   wherein a decrease in association of the reporter with the membrane following introduction of the test compound indicates that the test compound inhibits calcium ion influx.

In this aspect of the invention calcium influx into the cell is stimulated and then the cell is exposed to the test compound. In a preferred embodiment the cell is exposed to the test compound during calcium ion influx and the rate of dissociation of the reporter from the membrane verses the rate observed in a control cell(s) not exposed to the text compound is assessed.

Also provided is a method for identifying a compound that modulates calcium ion influx comprising:
(a) providing a eukaryotic cell having a detectable reporter capable of translocation from the cytosol to associate with the plasma membrane in response to an influx of calcium ions,
(b) providing a control by monitoring association of the reporter with the membrane while incubating the cell in conditions that stimulate calcium influx,
(c) monitoring association of the reporter with the membrane while
   (i) incubating the cell in the presence of a test compound, then
   (ii) incubating the cell in conditions that stimulate calcium influx, and
(d) comparing the association of the reporter with the membrane in the absence and presence of the test compound,
   wherein a change in association of the reporter with the membrane in the presence of the test compound compared to the association of the reporter with the membrane in the absence of the test compound indicates that the test compound modulates calcium influx.

In this aspect of the invention the cell is incubated with the test compound before calcium influx is stimulated.

Additionally the invention provides a method for identifying a compound that inhibits calcium ion influx comprising:
(a) providing a eukaryotic cell having a detectable reporter capable of translocation from the cytosol to associate with the plasma membrane in response to an influx of calcium ions,
(b) providing a control by monitoring association of the reporter with the membrane while incubating the cell in conditions that stimulate calcium influx,
(c) monitoring association of the reporter with the membrane while
   (i) introducing a test compound to the incubation mixture, then
   (ii) incubating the cell in conditions that stimulate calcium influx, and
(d) comparing the association of the reporter with the membrane in the absence and presence of the test compound
   wherein a decrease in the association of the reporter with the membrane in the presence of the test compound compared to the association of the reporter with the membrane in the absence of the test compound indicates that the test compound inhibits calcium influx.

In this aspect of the invention the cell is incubated with the test compound in the presence of calcium ions before calcium influx is stimulated.

In methods of the invention it is preferred that the cell (which may be a cell or cell line) is a mammalian cell, As used herein the term “cell” encompasses both cells (primary cells) and cell lines. Suitable cell lines include CHO, Cos, Jurkat-T, HeLa, PC12 or HEK293 cells, J774 mouse macrophage lines, B6MP102 mouse macrophage line, THP-1 monocyte-macrophage cell line, RAW264.7 macrophage cell line, HL-60 myeloid precursor cell line, PLB-986 promyelocytic cell line.

Examples of lymphocyte cell lines that can be used in a calcium influx assay according to the invention include T-lymphocyte cell lines such as Jurkat (human), or a B-lymphocyte cell lines such as A 20 (murine) or Raji (Human). Assays may also be performed on T-lymphocytes and/or B-lymphocytes isolated from human subjects, e.g. patients will allergic or autoimmune conditions. Traditionally, modulation of lymphocyte activity has been assessed using methods that measure cytokine production or assess lymphocyte proliferation. However, methods of the invention for detection of calcium influx are useful in novel assays for assessing the activity of test ligands at antigen receptors, such as the T-cell receptor (TCR) on T-lymphocytes or the B-cell receptor (BCR) on B-lymphocytes. Such methods can be employed in assays to detect ligands that are agonists, i.e. activatory compounds that stimulate calcium influx, such as an antigen e.g. a foreign or self antigen, a peptide antigen or antigenic epitope or mimic thereof, vaccine component and/or adjuvant. Antagonists, i.e. ligands that are inhibitory compounds which suppress or abolish calcium influx can also be detected using assays incorporating methods of the invention, these antagonists are useful as therapeutic agents for preventing or reversing auto-immunity in a subject. Methods of the invention can be used in assays for measuring the activity of altered peptide ligands (APL), such as agonist, partial agonist or antagonist peptides on T lymphocytes. Methods of the invention are advantageous as results can be rapidly generated, whereas current methods, such as cytokine secretion or lymphocyte proliferation assays for assessing lymphocyte activation generally take two to three days to perform.

In the methods of the invention, suitable conditions for incubation of the cells are those conditions generally used for whole cell assays. For example in specific embodiments, COS-7, HEK293 and HeLa cells can be transiently transfected with GFP-CAPRI plasmid DNA using Lipofectamine (GIBCO BRL) or GeneJuice transfection reagent (Merck) according to manufacturers instructions. After 24 hrs the cells can be imaged in EM buffer (121 mM NaCl, 5.4 mM KCl, 1.6 mM MgCl2, 6 mM NaHCO3, 9 mM glucose, 1.3 mM CaCl2, 25 mM HEPES, pH 7.4) or KH buffer (10 mM HEPES, 118 mM NaCl, 4.7 mM KCl, 10 mM glucose, 1.2 mM KH₂PO₄, 4.2 mM NaHCO₃, 1.2 mM CaCl₂, pH 7.4). For Ca²⁺-free conditions CaCl₂ can be replaced with 0.5 mM EGTA. To deplete intracellular Ca²⁺ stores prior to addback of Ca²⁺-containing media, which leads to sustained SOCE across the plasma membrane and CAPRI translocation, cells are stimulated with 1-5 μM thapsigargin in 2 ml Ca²⁺-free media for 5 minutes (volumes relevant to 22 mm coverslips and holder). Addition of 5 ml of Ca²⁺-containing media causes sustained Ca²⁺ entry across the plasma membrane.

To stimulate calcium influx, an agonist such as histamine (HeLa; 1-100 μM)) or ATP (HeLa, HEK293 and COS; 50 μM) can be applied by bulk addition (rapid mixing of 5 ml of agonist in appropriate imaging buffer e.g. EM or KH buffer as above) added to 2 ml of buffer containing the coverslip with vacuum line attachment to maintain total volume of 2 ml). These agonists activate SOCE and/or NSOCE. Histamine stimulation of HeLa cells can lead to sustained (>15 minutes) translocation of GFP-CAPRI to the plasma membrane at supramaximal agonist doses.

Live imaging in low throughput format is suitably performed at 25° C. or 37° C. on an inverted microscope (Nikon) attached to a PerkinElmer LCI or RS confocal imaging system.

The present invention can be applied generally to whole cell functional assays for any calcium channel, or any receptor or ion channel that is coupled to a calcium channel. Suitably, a cell used in a method of the invention is capable of expressing, endogenously and/or ectopically, one or more of the calcium channels selected from the group comprising: TRP family channels, voltage-gated channels, ligand-gated channels, receptor-operated channels. mechanosensitive channels, temperature sensitive channels, redox sensitive channels, osmolarity sensitive channels. By “endogenously” it is meant that the channel(s) is/are normally encoded by the cell, i.e. that the channels are present in the native cell. By “ectopically” it is meant that the channel or channels is/are expressed from nucleic acid that has been introduced into the cell and is either stably integrated into the genome, or is present extrachromosomally, e.g. following transient transfection with a vector(s) comprising nucleic acid encoding the ion channel(s).

Cells used in the methods of the invention have a calcium influx sensitive detectable reporter. In certain embodiments of the invention, the detectable reporter is a protein expressed within the cell. The cell can be engineered to express the detectable reporter protein from stably integrated nucleic acid or, for ectopic expression, the cell can be stably or transiently transfected with nucleic acid encoding the detectable reporter protein, suitably the nucleic acid encoding the detectable reporter protein is comprised within an expression vector.

In alternative embodiments of the methods, the cells do not express the calcium influx sensitive detectable reporter and instead, the reporter is introduced into the cell for purposes of conducting the assay, e.g. by permeabilisation, by using lipid reagents, or microinjection. This embodiment can be useful when using cells that endogenously or ectopically express an ion channel(s) or a receptor(s) desirable for use in the assay. Thus, as an alternative to expressing the detectable reporter within the cell, the detectable reporter can be introduced into the cell.

In preferred methods, the detectable reporter is a calcium influx specific reporter labelled with a fluorescent marker.

A suitable detectable reporter for use in methods in which the reporter is expressed within the cell, or in methods in which the reporter is introduced into the cell, is a protein chimera having a calcium influx sensitive reporter moiety and a fluorescent protein moiety.

The fluorescent protein can be a red, orange, yellow, yellow-green, green-yellow, green or blue (cyan) fluorescent protein. The fluorescent protein can be a wild type, enhanced, destabilised enhanced, or red-shift fluorescent protein.

As an alternative to using a single fluorescent protein marker system, a fluorescence resonance energy transfer (FRET) method using two fluorescent proteins may be used to detect the location of the reporter within the cell, e.g. FRET between a plasma membrane-localised fluorophore and a calcium influx sensitive fluorescent reporter according to the invention, e.g. a targeted CFP having a lipid group or transmembrane protein for targeting to the membrane and having a YFP-CAPRI or CAPRI derivative reporter, the YFP-CAPRI can be endogenously or ectopically expressed within the cell or introduced into the cell. Other suitable detectable reporters are those in which a calcium influx sensitive reporter protein moiety is either expressed within the cell, or introduced into the cell and the reporter is labelled in vivo, i.e. within the cell, with a fluorescent moiety which is introduced into the cell. Such detectable reporters include: a detectable reporter in which the C-terminus of the reporter is fused to ^{W160}hAGT (O⁶-alkylguanine-DNA alkyltransferase) which is fluorescently labelled following a reaction with O⁶-benzylguanine fluorescein (BGFL); and a detectable reporter in which the reporter a tetracysteine motif is added to the N- or C-terminus of the reporter and to which a bi-arsenic fluorophore is covalently linked (‘FIAsH labelling’).

Other fluorescent detectable reporters cannot be expressed within the cell, but are suitable for use in methods of the invention in which the calcium influx sensitive detectable reporter is introduced into the cell for example a detectable reporter in which the reporter protein is labelled with a fluorophore, such as a small organic fluorophore, e.g. fluorescein or rhodamine; or a quantum dot (Q dot).

In preferred embodiments of the invention, the reporter is labelled with a fluorescent marker which is a quantum dot (Quantum dot corporation). Quantum dots are tiny particles made from nanocrystal semiconductor materials, such as cadmium selenide. Dots of different sizes absorb UV light but then re-emit light at a different wavelength, usually at visible frequencies. The size of the dot determines the colour of light that it emits: a 2 nanometre dot emits green light, while a 5 nanometre dot emits red light. The reporter can be directly labelled with the quantum dot and introduced into the cell, alternatively the reporter can be labelled with the quantum dot by using a biotinylated reporter and streptavidin coated quantum dot both of which are introduced into the cell. As a further alternative using a quantum dot as the detectable marker, the reporter can be expressed within the cell as a chimera with avidin and a biotin-labelled quantum dot can be introduced into the cell.

In preferred methods the detectable reporter is a CAPRI reporter which is or comprises CAPRI, or a derivative thereof which is capable of association with the plasma membrane specifically in response to an influx of calcium ions, labelled with a detectable marker.

A CAPRI reporter according to present invention is generally applicable for detecting and measuring calcium influx of calcium whether by fluorescence, luminescent or other detection techniques, depending on the detectable marker employed.

In preferred methods of the invention, the detectable reporter is or comprises CAPRI, or a derivative thereof which is capable of association with the plasma membrane, labelled with a fluorescent protein, e.g. GFP-CAPRI.

A CAPRI derivative suitable for use in a method of the invention may be a mutated and/or truncated CAPRI that is capable of translocation to the plasma membrane specifically in response to an influx of calcium ions, labelled with a detectable marker. A CAPRI derivative is suitably a mutated CAPRI, having a mutation such that it no longer acts as a Ras GAP. A suitable mutant derivative of CAPRI is R473S [2], this derivative is advantageous because it translocates to the plasma membrane in response to Ca²⁺ influx but does not affect Ras, and thus its primary function is calcium influx sensing. In the R473S CAPRI mutant, arginine 473 has been substituted for serine, rendering the GAP function of CAPRI defective.

Other CAPRI derivatives useful as reporters in methods of the invention include mutated and/or truncated derivatives of CAPRI, e.g. those that increase or decrease the sensitivity to Ca²⁺ entry by altering the kinetics of association/dissociation of CAPRI at the membrane. It is important that CAPRI derivatives for use in methods of the invention retain the C2A C2B domains (tandem C2 domains) that primarily drive Ca²⁺-dependent association with the plasma membrane. Thus a further useful CAPRI derivative is a truncated derivative of CAPRI that is or comprises the tandem C2 domain C2A C2B, (Met 1 to Leu 275 or CAPRI), and in which the domain(s) may be in wild type or mutated form. Individually, the single domains of CAPRI have not been found to be suitable for use as reporters in methods of the invention, as neither the C2A (Met 1-Glu 141), nor the C2B domain (Glu 119-Leu 275) will translocate to the plasma membrane in response to an influx of calcium ions.

Fluorescent protein chimeras, i.e. fusion proteins, comprising a fluorescent protein, e.g. green fluorescent protein, and CAPRI, or a derivative thereof which is capable of association with the plasma membrane specifically in response to an influx of calcium ions, can be used as genetically-encodable detectable reporters of Ca²⁺ influx and are particularly suitable for use in methods of the invention.

Constructs encoding the CAPRI reporter can be transfected into cell lines by standard techniques e.g. electroporation, Ca²⁺ phosphate, lipofection, or by using a gene gun. Recombinant retroviruses, adenoviruses or lentiviruses can also be used to introduce genetic material encoding the CAPRI reporter into cells by infection. Selection of cells expressing a CAPRI reporter-fluorescent protein (CAPRI-FP) chimeric reporter can be made by FACS, or when a vector is used, a selectable marker carried by the vector, such as an antibiotic resistance gene, can provide a means for selection of transformed cells.

Alternatively a FRET method can be employed, in which, for example, the cell has a targeted cyan fluorescent protein (CFP) having a lipid group or transmembrane protein for targeting to the plasma membrane and has a yellow fluorescent protein (YFP) CAPRI or CAPRI derivative reporter, the YFP-CAPRI reporter can be expressed within the cell or introduced into the cell.

The invention thus provides a method for identifying a compound that modulates calcium ion influx comprising:
(a) providing a eukaryotic cell expressing FP-CAPRI or a derivative thereof which is capable of translocation to and association with the plasma membrane,
(b) incubating the eukaryotic cell in the presence of a test compound,
(c) monitoring fluorescence of the cell cytosol and/or plasma membrane,
   wherein a change in fluorescence in the cytosol and/or at the plasma membrane following addition of the test compound is indicative that the test compound modulates calcium ion influx.

The invention further provides a method for identifying a compound that stimulates (agonises) calcium ion influx comprising:
(a) providing a eukaryotic cell expressing FP-CAPRI or a derivative thereof, which is capable of translocation to and association with the plasma membrane,
(b) incubating the eukaryotic cell in the presence of a test compound,
(c) monitoring fluorescence of the cell cytosol, and/or plasma membrane
   wherein a decrease in cytosolic fluorescence, and/or increase in plasma membrane fluorescence following addition of the test compound is indicative that the test compound stimulates (agonises) calcium ion influx.

Also provided is a method for identifying a compound that inhibits (antagonises) calcium ion influx comprising:
(a) providing a eukaryotic cell expressing FP-CAPRI or a derivative thereof which is capable of translocation to and association with the plasma membrane,
(b) providing a stimulus for calcium ion influx,
(c) monitoring fluorescence of the cell cytosol and/or plasma membrane,
(d) introducing a test compound to the incubation mixture,
(e) monitoring fluorescence of the cell cytosol and/or plasma membrane,
   wherein an increase in cytosolic fluorescence and/or a decrease in plasma membrane fluorescence following addition of the test compound is indicative that the text compound inhibits (antagonises) calcium ion influx.

Further provided is a method for identifying a compound that agonises (stimulates) calcium ion influx comprising:
(a) providing a eukaryotic cell expressing FP-CAPRI or a derivative thereof which is capable of translocation to and association with the plasma membrane,
(b) incubating the eukaryotic cell in the presence of a test compound,
(c) monitoring fluorescence of the cell cytosol and/or plasma membrane,
   wherein a decrease in cytosolic fluorescence and/or an increase in plasma membrane fluorescence following addition of the test compound is indicative that the test compound agonises (stimulates) calcium ion influx.

An advantage of the methods of the invention is that the signal following calcium influx is prolonged, so that the signal can be readily and reliably detected by automated instrumentation.

The present invention advantageously provides for the use of instrumentation to detect fluorescent or luminescent signals from cells. In preferred methods the detectable marker is fluorescent and monitoring is performed by fluorescence microscopy. Fluorescence microscopy can be performed using wide-field or total internal reflection fluorescence microscopy (TIRF) or fluorescence lifetime imaging or confocal imaging.

In methods of the invention, cells having the fluorescent reporter can be imaged live, or fixed at a given time point, but preferably cells are imaged live, cells are preferably monitored using a HT imaging device, e.g. an Amersham INcell analyzer.

In methods of the invention, monitoring can be performed by measuring the fluorescence at the region(s) of interest within the cell over time. Association of the reporter with the plasma membrane and/or decrease of reporter in the cytosol can be assessed using any suitable algorithm or equation, an example is by calculating the relative translocation parameter (1-Ft/Fo) at one or more time points, wherein Fo is the fluorescence in a region(s) of interest (e.g. cytosol and/or plasma membrane) at the start of monitoring and Ft is fluorescence in a region(s) of interest at a later time point or points.

During monitoring, readings should be made as often as possible, ideally at intervals of less than 10 seconds; the length of time over which monitoring is performed will vary with the nature of the channel being analysed. Monitoring may be performed for time periods of from several seconds, to up to an hour. Suitably, readings may be taken every 5, 10, 15, 20 or 30 seconds over time periods of 5, 10, 20, 30 or 60 minutes. The frequency of readings and time period for monitoring can be experimentally determined and readily optimised for a particular assay, i.e. for particular cells/ion channels. For example, using HeLa cells stimulated with histamine, readings are taken every 4 or 5 seconds for up to 15 minutes.

Monitoring can be performed by measuring cytosolic fluorescence over time as assessed by calculating the relative translocation parameter at one or more time points, 1-Ft_{cyt}/Fo_{cyt}, wherein Fo_{cyt} is the cytosolic fluorescence in the region of interest at the start of monitoring and Ft_{cyt} is the cytosolic fluorescence in the region of interest at a particular time point. A decrease in cytosolic fluorescence results in an increase in the relative translocation parameter.

Methods of the invention can be performed in high throughput format.

Many functional assays for calcium signalling are termed “flash” assays because they are instantaneous and transient as they are complete within a few seconds of the calcium ion influx stimulus, however in methods of the invention calcium ion influx can be detected for prolonged periods of tens of minutes up to several hours.

The invention is advantageously employed in whole cell functional assays and high throughput screening assays for calcium signalling to extend the signal so that it can be read by conventional plate readers.

Methods of the invention are ideal for high throughput screening of calcium influx modulators, for example in 96, 384, or 3456 multiwell plates or other plate formats. In a preferred embodiment, the assay is conducted in a multi well plate format and an instrument is used for monitoring in each well. The multiwell plates can be handled over an extended time period because of the extended time period over which calcium influx can be detected.

The invention provides methods for identification of calcium channel modulating compounds, preferably in high throughput screening (HTS) format, using cells having a reporter derived from the protein CAPRI (e.g. fluorescent protein tagged CAPRI or CAPRI derivative) which is expressed by the cell or introduced into the cell.

In a method of the invention, cells expressing a fluorescent protein-CAPRI reporter (FP-CAPRI), or a derivative thereof are incubated in the presence of a test compound, the effect of the test compound on calcium channels (block/activation) can be detected using an FP-CAPRI reporter as a reporter for calcium influx; with translocation of the CAPRI reporter to the membrane and maintenance at that locus being specifically indicative of calcium ion influx. To detect compounds that block influx, the cells are subjected to a calcium influx stimulus, which may be provided before, during and/or after exposure of the cell to the test compound.

In preferred method of the invention an FP-CAPRI reporter, e.g. GFP-CAPRI, is genetically encoded, and cell lines of interest can be cloned to stably express the reporter, allowing consistency between experiments. Translocation is specifically sensitive to Ca²⁺ influx across the plasma membrane, so the FP-CAPRI detectable reporter is a sensitive and specific indicator of Ca²⁺ channel activation/block.

Cells having the fluorescent reporter are imaged fixed at a given time point, or preferably, live. For imaging, the use of an HT imaging device, e.g. an Amersham INcell analyzer, is preferred.

The methods of the invention are ideal for use in HTS to identify new calcium ion channel blockers. The method can be performed using currently available multi-well imaging platforms.

Methods of the invention are usually performed such that the cells are incubated throughout the method in media containing calcium ions. However, in some embodiments of methods of the invention cells are initially incubated in calcium-free media and in the presence of a compound that enhances the release and/or leak of calcium from the intracellular store prior to exposure of the cells to the test compound and/or calcium influx stimulus; in methods that employ this treatment, calcium is then added back to the incubation so that influx can be detected.

### LIST OF FIGURES

FIG. 1, Molecular architecture of CAPRI and RASAL, with percentage identity between RASAL and CAPRI indicated. Each protein consists of C2A and C2B domains, a central GAP-related domain (GRD), and a pleckstrin homology (PH), Tec kinase domain (Btk).

FIG. 2A, Relative translocation parameter. Each image was background corrected prior to measuring the average pixel intensity of a given region of interest (ROI) corresponding to >10% of the cytosolic area. The Relative Translocation parameter was calculated as shown, wherein F₀=maximum cytosolic fluorescence, Fₜ=cytosolic fluorescence at given time point and the Relative Translocation=1-Ft/Fo. The Relative Translocation (RT) is an indication of the translocation of a given GFP chimera to the plasma membrane.

FIG. 2B, Translocation of CAPRI and RASAL in response to 100 μM histamine stimulation of HeLa cells. Time (t)=seconds after histamine stimulation. Translocation of GFP-CAPRI is sustained whereas GFP-RASAL is transient and oscillatory in parallel experiments. GFP-CAPRI is detectable at the plasma membrane for up to 15 minutes. This was the maximum length of experimental monitoring performed, taking into account photobleaching of GFP with an acquisition rate of 15 frames/second, 2×2 binning, and an exposure time of 1 second per image on the PerkinElmer RS confocal microscope.

FIG. 2C Kinetics of CAPRI and RASAL translocations. RASAL (bottom) oscillates between the plasma membrane and cytosol in phase with cytosolic Ca²⁺ oscillations [4]. Average n=3 cells. In contrast, CAPRI (top) never oscillates in phase with repetitive Ca²⁺ signals in the cytosol. Instead, translocation to the plasma membrane is sustained. Average n=7 cells from n=6 experiments for 100 μM histamine, n=5 cells from n=3 experiments for 10 μM histamine.

FIG. 2D CAPRI and RASAL translocations in COS-7 cells stimulated with 50 μM ATP. The rate of RASAL translocation matches the release of Ca²⁺ from intracellular stores. In contrast the rate of CAPRI translocation (grey line) is slow and more sustained. This is likely to reflect the fact that CAPRI, operating as a Ca²⁺ influx sensor, is sensitive to the latency of SOCE/NSOCE induced by purinergic stimulation in this cell type. Experiment is an average of n=6 cells for GFP-CAPRI and n=9 cells from 2 experiments for GFP-RASAL. Methods as for Example 1.

FIG. 3 Representative traces of sequential Fura-2 and GFP imaging of HeLa cells expressing GFP-CAPRI. Fura-2 emission was monitored at 380 nm excitation only (non-ratiometric) to track changes in cytosolic Ca²⁺ concentration, expressed as the inverse change in fluorescence intensity at 380 nm excitation (black line). GFP-CAPRI translocation (grey line) is expressed as the Relative Translocation according to FIG. 2A. 10 μM histamine application generates sinusoidal Ca²⁺ oscillations or base-line Ca²⁺ spikes. GFP-CAPRI is refractory to repetitive Ca²⁺ signals unlike RASAL [4] which is entirely consistent with the preferential sensing of Ca²⁺ entry by CAPRI.

FIG. 4 CAPRI sensitivity to SOCE. GFP-CAPRI (bold line), GFP-RASAL (thin line) and GFP-PKCγ (doffed line) expressing HeLa cells were treated with 1 μM thapsigargin in 2 ml KH media containing no added CaCl₂ and 0.5 mM EGTA (nominally zero Ca²⁺-containing media) for 15 minutes. At a time point 20 seconds after the initiation of imaging, 5 ml of KH media+1 μM thapsigargin in 1.3 mM CaCl₂ KH buffer was added by bulk addition. CAPRI association with the membrane is maintained by store-operated Ca²⁺ entry through SOCE channels. Methods as for Example 1. Averages of n=16 cells, n=2 experiments for PKC, n=7 n=4 for CAPRI, n=13 n=3 for RASAL.

FIG. 5 Representative CAPRI, RASAL and PKCgamma translocations as measured by Total Internal Reflection Fluorescence Microscopy (TIRFM). HeLa cells expressing GFP CAPRI were imaged as in Example 1, but using an Olympus Cell TIRF system using 473 nm laser excitation and a 60×TIRFM objective, 2×2 binning at approximately 4 frames/sec. For calculation of the relative translocation parameter the average pixel intensity of a given region of interest (ROI) corresponding to >10% of the membrane area was used. The Relative Translocation (RT) parameter was calculated, RT=1-Ft/F0, to provide an indication of the translocation of the GFP fusion proteins to the plasma membrane. CAPRI translocation to the membrane is sustained and does not oscillate in contrast to PKCγ and RASAL.

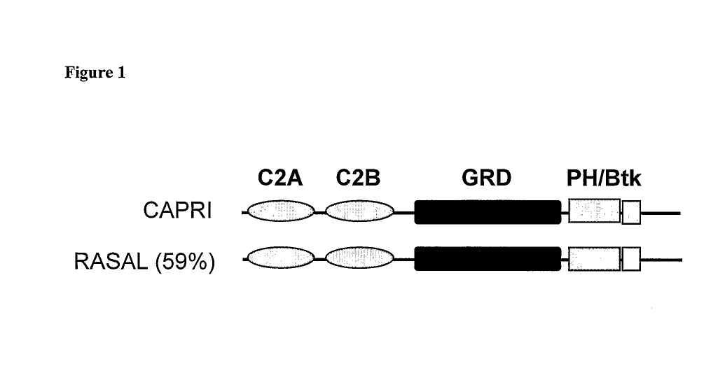

### REFERENCES

[1] Walker, S. A., Cullen, P. J., Taylor, J. A., and Lockyer, P. J. Control of Ras cycling by Ca²⁺. FEBS Letters (2003) 546, 6-10.
[2] Lockyer, P. J. et al., CAPRI regulates Ca²⁺-dependent inactivation of the Ras-MAPK pathway. Curr. Biol. 11, 981-86 (2001).
[3] Allen M. et al., Gene 218 (1998) 17-25
[4] Walker, S. et al. (2004) EMBO J. in press.

### EXAMPLES

### Example 1

### Ca²⁺ Influx Assay Using a Genetically-Encoded Fluorescent Reporter—Translocation of CAPRI and RASAL in Response to Agonist (100 μM Histamine) Stimulation of HeLa Cells

The day before transfection HeLa cells were seeded onto 22 mm glass coverslips in 6-well tissue culture dishes at such a density to reach 60-80% confluence within 24 hours. The following day (approximately 24 hours later) HeLa cells were transfected with green fluorescent protein GFP-CAPRI or GFP-RASAL using GeneJuice Transfection Reagent (Merck) according to the manufacturers instructions.

After 24 hrs incubation in transfection mix (complete media plus transfection reagent) at 37° C., 5% CO₂) coverslips were transferred to holders containing 2 ml of KH buffer (10 mM HEPES, 118 mM NaCl, 4.7 mM KCl, 10 mM glucose, 1.2 mM KH₂PO₄, 4.2 mM NaHCO₃, 1.2 mM CaCl₂, pH 7.4) and monitoring performed by live imaging in a 37° C. heated chamber on an inverted Nikon TE-2000 microscope using a 40× oil objective lens. Individual images were acquired every 4 seconds for up to 15 minutes with an exposure time of 1 sec, 2×2 binning, using a PerkinElmer RS confocal system with a 488 nm laser line.

Rapid mixing of the agonist histamine was achieved by bulk addition of 5 ml of 37° C. KH media plus histamine, with a vacuum line maintaining a maximum volume of 2 ml within the coverslip holder.

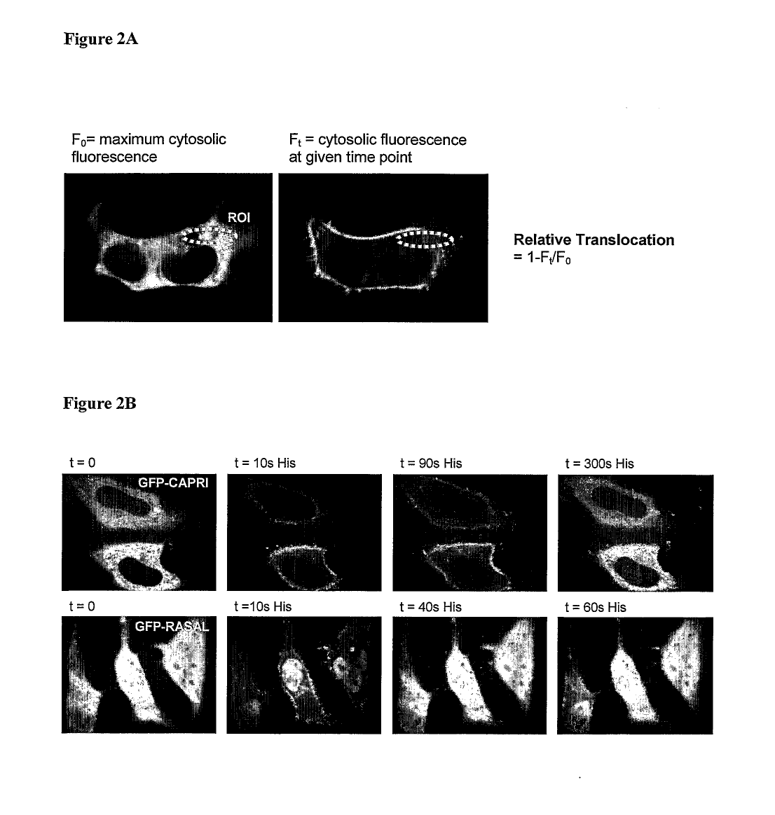

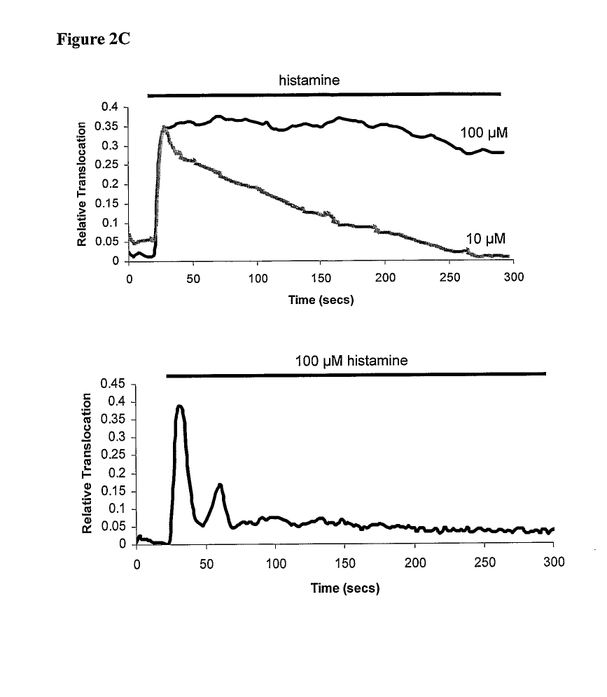

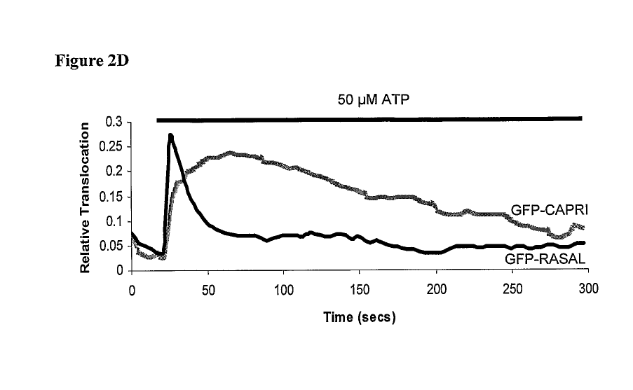

For calculation of the relative translocation parameter, each image was background corrected prior to measuring the average pixel intensity of a given region of interest (ROI) corresponding to >10% of the cytosolic area (see FIG. 2A). The Relative Translocation (RT) parameter was calculated, RT=1-Fₜ/F₀, to provide an indication of the translocation of the GFP-CAPRI or GFP-RASAL chimera to the plasma membrane,

Translocation of CAPRI and RASAL in response to agonist stimulation of calcium influx into HeLa cells using 100 μM histamine is shown in FIG. 2B. Time (t)=seconds after histamine stimulation. Translocation of GFP-CAPRI and maintenance at the plasma membrane is sustained, whereas GFP-RASAL is transient and oscillatory in parallel experiments. GFP-CAPRI is detectable at the plasma membrane for up to 15 minutes following stimulation of influx. This was the maximum length of the experiment taking into account photobleaching of GFP with an acquisition rate of 15 frames/second, 2×2 binning, and an exposure time of 1 second per image on the PerkinElmer RS confocal microscope.

The kinetics of CAPRI and RASAL translocations are shown in FIG. 2C in which the relative translocation parameter is plotted against time. RASAL (bottom) oscillates between the plasma membrane and cytosol in phase with cytosolic Ca²⁺ oscillations [4]. Average n=3 cells. In contrast, CAPRI (top) never oscillates in phase with repetitive Ca²⁺ signals in the cytosol. Instead, translocation to the plasma membrane is sustained. Average n=7 cells from n=6 experiments for 100 μM histamine, n=5 cells from n=3 experiments for 10 μM histamine.

CAPRI and RASAL translocations in COS-7 cells stimulated with 50 μM ATP (to release calcium ions from the intracellular store) are shown in FIG. 2D. The rate of RASAL translocation matches the release of Ca²⁺ from intracellular stores. In contrast the rate of CAPRI translocation (grey line) is slow and more sustained. This indicates that CAPRI, operating as a Ca²⁺ influx sensor, is sensitive to the latency of SOCE/NSOCE induced by purinergic stimulation in this cell type. The experiment is an average of n=6 cells for GFP-CAPRI and n=9 cells from 2 experiments for GFP-RASAL. The methods used were as described above.

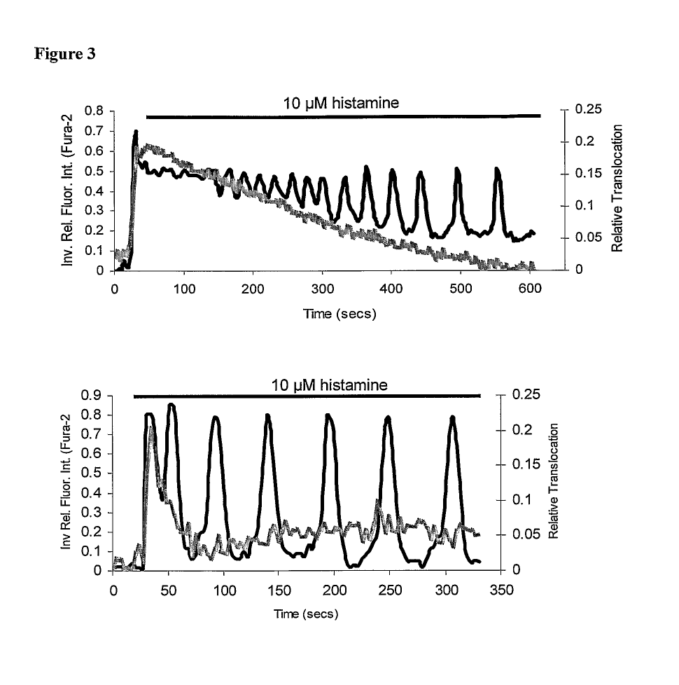

Representative traces of sequential Fura-2 and GFP imaging of HeLa cells expressing GFP-CAPRI over 600 seconds (top) and 330 seconds (bottom) are shown in FIG. 3. Fura-2 emission was monitored at 380 nm excitation only (non-ratiometric) to track changes in cytosolic Ca²⁺ concentration, expressed as the inverse change in fluorescence intensity at 380 nm excitation (black line). GFP-CAPRI translocation (grey line) is expressed as the Relative Translocation calculated as above. Application of 10 μM histamine generates sinusoidal Ca²⁺ oscillations or base-line Ca²⁺ spikes. GFP-CAPRI is refractory to repetitive Ca²⁺ signals unlike RASAL [4] which is entirely consistent with the preferential sensing of Ca²⁺ influx by CAPRI.

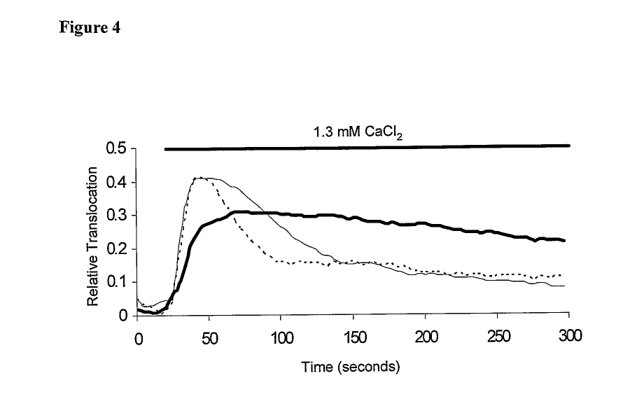

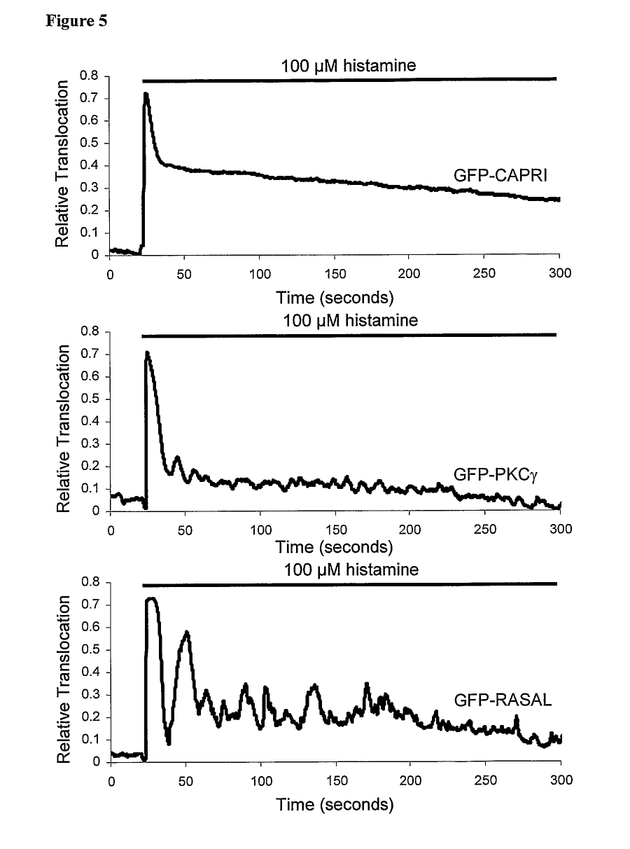

CAPRI sensitivity to SOCE is shown in FIG. 4. In this addback protocol to demonstrate SOCE, the methods used were as given above, except that GFP-CAPRI (black trace) and GFP-RASAL (grey trace) expressing HeLa cells were first treated with 1 μM thapsigargin in 2 ml KH media containing no added CaCl₂ and 0.5 mM EGTA (nominally zero Ca²⁺-containing media); then, after 40 seconds, 5 ml of KH media/1 μM thapsigargin (1.3 mM CaCl₂ KH buffer) was added by bulk addition, providing Ca²⁺ ions. Cells were monitored as before and readings taken every 5 seconds for up to 15 minutes. In the period that RASAL takes to fully dissociate from the membrane CAPRI association is maintained by store-operated Ca²⁺ entry through SOCE channels. Thapsigargin selectively blocks release of calcium ions from the intracellular store (CICR).

### Example 2

### Screening to Detect a Receptor Antagonist Compound

The day before transfection COS-7, HEK293 and HeLa cells are seeded onto 22 mm glass coverslips in 6-well tissue culture dishes at such a density to reach 60-80% confluence within 24 hours. After approximately 24 hours, COS-7, HEK293 and HeLa cells are transiently transfected with GFP-CAPRI plasmid DNA using Lipofectamine (GIBCO BRL) or GeneJuice transfection reagent (Merck) according to manufacturers instructions and incubated for 24 hours as above. The cells are incubated with test compound in EM buffer (121 mM NaCl, 5.4 mM KCl, 1.6 mM MgCl2, 6 mM NaHCO3, 9 mM glucose, 1.3 mM CaCl2, 25 mM HEPES, pH 7.4) or KH buffer (10 mM HEPES, 118 mM NaCl, 4.7 mM KCl, 10 mM glucose, 1.2 mM KH₂PO₄, 4.2 mM NaHCO₃, 1.2 mM CaCl₂, pH 7.4) for up to one hour.

To stimulate calcium influx, the agonist histamine (HeLa; 1-100 μM)) or ATP (HeLa, HEK293 and COS; 50 μM) is applied by bulk addition (rapid mixing of 5 ml of agonist in appropriate imaging buffer e.g. EM or KH buffer as above) to 2 ml of the corresponding EM or KH buffer containing the coverslip with vacuum line attachment to maintain total volume of 2 ml.

The cells are monitored before during and after stimulation of calcium influx using a multi-well high throughput imaging system (INcell Analyzer, Amersham Biosciences. Inhibition of CAPRI translocation to the plasma membrane is indicative of histamine receptor blockade, or blockade of a secondary pathway downstream of the GPCR but upstream of Ca²⁺ mobilisation, indicating that the test compound is a calcium influx antagonist.

For low throughput screening, monitoring is performed on the cells before, during and after application of the stimulus for calcium influx by live imaging at 25° C. or 37° C. on an inverted microscope (Nikon) attached to a PerkinElmer LCI or RS confocal imaging system.

### Example 3

### Screening for SOCE Channel Inhibitors

The day before transfection COS-7, HEK293 and HeLa cells are seeded onto 22 mm glass coverslips in 6-well tissue culture dishes at such a density to reach 60-80% confluence within 24 hours. After approximately 24 hours, COS-7, HEK293 and HeLa cells are transiently transfected with GFP-CAPRI plasmid DNA using Lipofectamine (GIBCO BRL) or GeneJuice transfection reagent (Merck) according to manufacturers instructions and incubated for 24 hours as above.

Prior to introduction of the test compound, cells are stimulated with 1-5 μM thapsigargin in 2 ml Ca²⁺-free media for 5 minutes. The cells are then incubated in the presence of the test compound for up to one hour.

The cells are imaged in Ca²⁺-free EM buffer (121 mM NaCl, 5.4 mM KCl, 1.6 mM MgCl2, 6 mM NaHCO3, 9 mM glucose, 0.5 mM EGTA, 25 mM HEPES, pH 7.4) or Ca²⁺-free KH buffer (10 mM HEPES, 118 mM NaCl, 4.7 mM KCl, 10 mM glucose, 1.2 mM KH₂PO₄, 4.2 mM NaHCO₃, 0.5 mM EGTA, pH 7.4). before and during and after Ca²⁺ addback, by bulk addition of 5 ml of EM or KH buffer containing Ca²⁺ to the 2 ml of corresponding Ca²⁺-free buffer containing the coverslip. A vacuum line attachment was used to maintain total volume of 2 ml.

Live imaging was performed at 25° C. or 37° C. before, during and after Ca²⁺ addback on an inverted microscope (Nikon) attached to a PerkinElmer LCI or RS confocal imaging system. Readings are taken every 5 seconds for 15 minutes.

Failure to of translocation of the CAPRI reporter, or reduced translocation of the CAPRI reporter indicates Ca²⁺ channel inhibition by the test compound.

### Example 4

### Screening for SOCE Channel Inhibitors

The day before transfection COS-7, HEK293 and HeLa cells are seeded onto 22 mm glass coverslips in 6-well tissue culture dishes at such a density to reach 60-80% confluence within 24 hours. After approximately 24 hours, COS-7, HEK293 and HeLa cells are transiently transfected with GFP-CAPRI plasmid DNA using Lipofectamine (GIBCO BRL) or GeneJuice transfection reagent (Merck) according to manufacturers instructions and incubated for 24 hours as above.

The cells are incubated with 1-5 μM thapsigargin in 2 ml Ca²⁺-free EM or KH media for 5 minutes. Ca²⁺ addback is then carried out by bulk addition of 5 ml of EM or KH buffer (as appropriate) containing Ca²⁺ to the 2 ml of corresponding Ca²⁺-free buffer containing the coverslip. A vacuum line attachment was used to maintain total volume of 2 ml.

The test compound is added to cells 100 seconds after Ca²⁺ addback by bulk addition of 5 ml of EM or KH buffer as appropriate containing the test compound.

Live imaging is performed at 25° C. or 37° C. before, during and after addition of the test compound using an inverted microscope (Nikon) attached to a PerkinElmer LCI or RS confocal imaging system. Readings are taken every 5 seconds for 15 minutes.

An increase in the rate of GFP-CAPRI dissociation from the membrane compared to control cells indicates the effectiveness of Ca²⁺ entry blockade by the test compound.

### Sequence Information

Genebank accession numbers for CAPRI (RASA4) are NM_{—}006989 and AY029206. Genebank accession number for RASAL is NM_{—}004658.

What is claimed is:

1. A method for identifying a compound capable of modulating influx of calcium ions into a eukaryotic cell comprising:
(a) providing a eukaryotic cell having a detectable reporter capable of translocation from the cytosol to associate with the plasma membrane in response to an influx of calcium ions,
(b) incubating the cell with a test compound,
(c) providing a stimulus for calcium influx, and,
(d) monitoring association of the detectable reporter with the plasma membrane and/or a decrease in the detectable reporter in the cytosol,
wherein the stimulus for calcium influx is provided before during and/or after before during, and/or after incubation of the cell with the test compound.

2. The method according to claim 1 comprising:
(a) monitoring association of the detectable reporter with the membrane and/or loss of the detectable reporter from cytosol while
(i) providing a stimulus for calcium influx by incubating the cell in conditions that stimulate calcium influx, then
(ii) providing a test compound and incubating the cell with the test compound, and,
(b) comparing the association of the detectable reporter with the membrane and/or loss of the detectable reporter from the cytosol in the absence of the test compound with the association of the detectable reporter with the membrane and/or loss of the detectable reporter from the cytosol in the presence of the test compound,
wherein a difference in the association of the detectable reporter with the membrane and/or loss of detectable reporter from the cytosol in the presence of the test compound indicates that the test compound modulates calcium influx.

3. The method according to claim 2, for identifying a compound that antagonises calcium ion influx comprising:
(a) monitoring association of the detectable reporter with the membrane and/or loss of the detectable reporter from the cytosol while
(i) providing a stimulus for calcium influx by incubating the cell in conditions that stimulate calcium influx, then,
(ii) providing a test compound and incubating the cell with the test compound, and,
(b) comparing the association of the detectable reporter with the membrane and/or loss of detectable reporter from the cytosol in the absence and presence of the test compound,
wherein a decrease in association of the detectable reporter with the membrane and/or an increase in the detectable reporter in the cytosol following incubation of the cell with the test compound indicates that the test compound antagonises calcium ion influx.

4. The method according to claim 1 for identifying a compound that modulates calcium ion influx comprising:
(a) providing a control by monitoring association of the detectable reporter with the membrane and/or loss of the detectable reporter from the cytosol while incubating the cell in conditions that stimulate calcium influx,
(b) monitoring association of the detectable reporter with the membrane and/or loss of the detectable reporter from the cytosol while
(i) incubating the cell with a test compound, then
(ii) providing a stimulus for calcium influx by incubating the cell in conditions that stimulate calcium influx, and
(c) comparing the association of the detectable reporter with the membrane and/or loss of the detectable reporter from the cytosol in the absence and presence of the test compound,
wherein a difference in association of the detectable reporter with the membrane and/or a difference in loss of the detectable reporter from the cytosol in the presence of the test compound compared to that in the absence of the test compound indicates that the test compound modulates calcium influx.

5. The method according to claim 4 for identifying a compound that antagonises calcium ion influx comprising:
(a) providing a control by monitoring association of the detectable reporter with the membrane and/or loss of the detectable reporter from the cytosol while providing a stimulus for calcium influx by incubating the cell in conditions that stimulate calcium influx,
(b) monitoring association of the detectable reporter with the membrane and/or loss of the detectable reporter from the cytosol while
(i) incubating the cell with a test compound, then
(ii) providing a stimulus for calcium influx by incubating the cell in conditions that stimulate calcium influx, and
(c) comparing the association of the detectable reporter with the membrane and/or loss of the detectable reporter from the cytosol in the absence and presence of the test compound,
wherein a decrease in the association of the detectable reporter with the membrane and/or a decrease in loss of the detectable reporter from the cytosol in the presence of the test compound compared to that in the absence of the test compound indicates that the test compound antagonises calcium influx.

6. The method according to claim 1, for identifying a compound that agonises calcium ion influx comprising:
(a) providing a eukaryotic cell having a detectable reporter capable of translocation from the cytosol to associate with the plasma membrane in response to an influx of calcium ions,
(b) providing a control by monitoring association of the detectable reporter with the membrane and/or loss of the detectable reporter from the cytosol while incubating the cell in conditions that stimulate calcium influx,
(c) monitoring association of the detectable reporter with the membrane and/or loss of the detectable reporter from the cytosol while
(i) providing a test compound and incubating the cell with the test compound, then,
(ii) providing a stimulus for calcium influx by incubating the cell in conditions that stimulate calcium influx, and,
(d) comparing the association of the detectable reporter with the membrane and/or loss of the detectable reporter from the cytosol in the absence and presence of the test compound,
wherein an increase in the association of the detectable reporter with the membrane and/or an increase in loss of detectable reporter from the cytosol in the presence of the test compound compared to that in the absence of the test compound indicates that the test compound agonises calcium influx.

7. A method for identifying a compound that agonises calcium ion influx comprising:
(a) providing a eukaryotic cell having a detectable reporter capable of translocation from the cytosol to associate with the plasma membrane in response to an influx of calcium ions,
(b) monitoring association of the detectable reporter with the membrane and/or loss of the detectable reporter from the cytosol before and after incubation of the cell with the test compound, and,
(c) comparing the association of the detectable reporter with the membrane and/or loss of the detectable reporter from the cytosol in the absence and presence of the test compound,
wherein an increase in association of the detectable reporter with the membrane and/or a decrease in the detectable reporter in the cytosol following introduction of the test compound indicates that the test compound agonises calcium ion influx.

8. The method according to claim 1, wherein the cell is a mammalian cell.

9. (canceled)

10. The method according to claim 1, wherein the cell is capable of expressing, endogenously or ectopically, one or more calcium channel selected from the group comprising: TRP family channels, voltage-gated channels, ligand-gated channels and receptor-operated channels.

11. The method according to claim 10, wherein the detectable reporter is expressed within the eukaryotic cell.

12. The method according claim 11, wherein a nucleic acid encoding the detectable reporter is stably integrated within the eukaryotic cell or is transiently transfected into the eukaryotic cell.

13. (canceled)

14. The method according to claim 1, wherein the detectable reporter is introduced into the cell.

15. The method according to claim 1, wherein the detectable reporter is or comprises CAPRI, or a derivative thereof which is capable of translocation to and association with the plasma membrane, and a detectable marker.

16. The method according to claim 15, wherein the detectable reporter is or comprises the CAPRI derivative R473S, and a detectable marker.

17. The method according to claim 15, wherein the detectable reporter is a CAPRI derivative comprising or consisting of the CAPRI C2A C2B domains, with wild type or mutated sequence, and a detectable marker.

18. The method according to claim 1, wherein the detectable reporter is a reporter labelled with a fluorescent marker.

19. The method according to claim 1 for identifying a compound that modulates calcium ion influx comprising:
(a) providing a eukaryotic cell expressing FP-CAPRI or a FP-CAPRI derivative which is capable of translocation to and association with the plasma membrane;
(b) (i) as a control, incubating the eukaryotic cell in the absence of test compound then providing a stimulus for calcium influx, and,
(ii) incubating the eukaryotic cell in the presence of a test compound, then providing a stimulus for calcium influx; or;
(c) (i) as a control, incubating the eukaryotic cell and providing a stimulus for calcium influx in the absence of test compound, and,
(ii) incubating the eukaryotic cell and providing a stimulus for calcium influx, then providing a test compound; and,
(d) monitoring fluorescence of the cell cytosol and/or plasma membrane during incubation in (b) or (c),
wherein a difference in fluorescence in the cytosol and/or at the plasma membrane in the presence of the test compound compared with that in the absence of the test compound is indicative that the test compound modulates calcium ion influx.

20. The method according to claim 7 for identifying a compound that agonises calcium ion influx comprising:
(a) providing a eukaryotic cell expressing FP-CAPRI or a FP-CAPRI derivative, which is capable of translocation to and association with the plasma membrane,
(b) incubating the eukaryotic cell in the presence of a test compound, and,
(c) monitoring fluorescence of the cell cytosol, and/or plasma membrane,
wherein a decrease in cytosolic fluorescence, and/or increase in plasma membrane fluorescence following addition of the test compound is indicative that the test compound agonises calcium ion influx.

21. The method according to claim 1 for identifying a compound that antagonises calcium ion influx comprising:
(a) providing a eukaryotic cell expressing FP-CAPRI or a FP-CAPRI derivative which is capable of translocation to and association with the plasma membrane,
(b) incubating the eukaryotic cell and providing a stimulus for calcium ion influx,
(c) monitoring fluorescence of the cell cytosol and/or plasma membrane,
(d) introducing a test compound to the incubation mixture,
(e) monitoring fluorescence of the cell cytosol and/or plasma membrane,
wherein an increase in cytosolic fluorescence and/or a decrease in plasma membrane fluorescence following addition of the test compound is indicative that the test compound antagonises calcium ion influx.

22. The method according to claim 7 for identifying a compound that agonises calcium ion influx comprising:
(a) providing a eukaryotic cell expressing FP-CAPRI or a FP-CAPRI derivative which is capable of translocation to and association with the plasma membrane,
(b) incubating the eukaryotic cell in the presence of a test compound and,
(c) monitoring fluorescence of the cell cytosol and/or plasma membrane,
wherein a decrease in cytosolic fluorescence and/or an increase in plasma membrane fluorescence following addition of the test compound is indicative that the test compound agonises calcium ion influx.

23. The method according to claim 19, wherein the fluorescent marker is selected from the group consisting of a quantum dot and a fluorescent protein.

24-27. (canceled)

28. The method according to claim 19, wherein monitoring is performed by fluorescence microscopy.

29-30. (canceled)

31. The method according to claim 28, wherein monitoring association of the detectable reporter with the plasma membrane and/or decrease of detectable reporter in the cytosol is assessed by calculating the relative translocation parameter (1-Ft/Fo) at one or more time points, wherein Fo is the fluorescence in a region(s) of interest at the start of monitoring and Ft is fluorescence in a region(s) of interest at a later time point or points.

32-38. (canceled)
